(19) United States
(12) Patent Application Publication
(10) Pub. No.: US 2002/0192274 A1
(43) Pub. Date: Dec. 19, 2002

(54) pH sensitive liposomal drug delivery

(76) Inventor: Biddanda C. Ponnappa, Brookhaven, PA (US)

Correspondence Address: DRINKER BIDDLE & REATH, ONE LOGAN SQUARE, 18TH AND CHERRY STREETS, PHILADELPHIA, PA, 19103-6996, US

(21) Appl. No.: 10/106,142

(22) Filed: Mar. 25, 2002

Related U.S. Application Data
(60) Provisional application No. 60/278,605, filed on Mar. 26, 2001.

(51) Int. Cl. A61K 9/127

(52) U.S. Cl. 424/450

(57) ABSTRACT

The present invention discloses a novel liposome composition wherein phosphatidyl ethanolamine, cholesteryl hemisuccinate, and cholesterol in a ratio of 7:4:2 allow for the efficacious administration of a therapeutic agent to a macrophage. The liposomes of the present invention are stable at physiological pHs, while at the same time being fusogenic at acidic pHs. This property allows for the delivery of the therapeutic agent into the cytosol, and subsequently the nucleus, of the macrophage. The liposome composition disclosed herein is useful in the treatment of macrophage associated diseases or conditions.

### CROSS REFERENCE TO RELATED APPLICATIONS

This application claims priority under 35 U.S.C.§119 based upon U.S. Provisional Patent Application No. 60/278,605, filed Mar. 26, 2001.

### GOVERNMENT RIGHTS IN THE INVENTION

This invention was made with government support under grants AA10967 and AA07186 awarded by the National Institute of Health. The government has certain rights in the invention.

### FIELD OF THE INVENTION

The present invention relates to the fields of lipid chemistry and cell biology, and to a method of treating a macrophage associated disease and, more particularly, to anionic liposomes for delivering a therapeutic agent to macrophages.

### BACKGROUND OF THE INVENTION

When activated under physiologically challenging conditions, such as endotoxemia or immune reactions, macrophages release large amounts of cytokines, interleukins and prostanoids, which may result in organ damage. Kupffer cells, the resident macrophages in the liver, play a major role in the pathogenesis of liver injury. It has been demonstrated that obliteration of Kupffer cells prior to administration of hepatotoxins prevents liver damage. (Adachi, Y., et al., Hepatology 20: 453-460, 1994; Laskin, D. L., et al, Hepatology 21: 1045-1050, 1995; Ishiyama, H., et al., Pharmacol. Toxicol. 77: 293-298, 1995). Tumor necrosis factor alpha (TNF-α), a pro-inflammatory cytokine, exhibits pleiotropic effects on various cell types. (Beutler, B., Cerami, A., Nature 320:, 584-588, 1986). Kupffer cells are the major producers of TNF-α following exposure to lipopolysaccharide (LPS), the bacterial endotoxin. (Decker, K., Eur. J. Biochem. 192: 245-261, 1990). An over-production of TNF-α has been associated with the development of alcoholic liver injury (McClain, C. J., Cohen, D. A., Hepatology 9: 349-351, 1989; Nanji, A. A., et al., Hepatology 19: 1483-1487, 1994; Kamimura, S., Tsukamoto, H., Hepatology 21: 1304-1309, 1995), rheumatoid arthritis (Elliot, M. J., et al., Lancet 344:, 1105-1110, 1994), inflammatory bowel disease (Miurch, S. H., et al., Gut 34: 1705-1709, 1993), and septic shock (Michie, H. R., et al., N. Engl. J. Med. 318: 1481-1486, 1988). Antibodies which bind TNF-α, neutralize the effects of TNF-α released by Kupffer cells in conditions such as ischemia reperfusion (Wanner, G. A., et al., Shock 11: 391-395, 1999) and experimental liver damage induced by chronic alcohol consumption. (Iimuro Y., et al., Hepatology 26: 1530-1537, 1997).

In recent years, the use of antisense oligodeoxynucleotides (ASOs) has been an alternative approach to suppress the synthesis of specific proteins. ASOs contain sequences complementary to RNAs, which block or destroy the targeted mRNAs (Matteucci, M. D., Wagner, R. W., Nature 384: S20-22, 1994; Tu, G-C., et al., J. Biol. Chem. 273: 25125-25131, 1998). Recently, a highly effective phosphorothioate-modified ASO was developed, TJU-2755 (Tu, G-C., et al., J. Biol. Chem. 273: 25125-25131, 1998), against rat TNF-α. (Tu, G-C., et al., J. Biol. Chem. 273: 25125-25131, 1998). While TJU-2755 (SEQ. ID. NO: 1) was highly effective in primary Kupffer cells ex vivo (>90% inhibition of LPS-stimulated TNF-α production), the in vivo efficacy has not been determined.

The in vivo efficacy of any drug depends upon the efficiency with which it is delivered to the relevant cellular compartment in the target cells. Recently, anionic liposomes, which entrap the ASO inside lipidic membranes, have been demonstrated to be an efficient delivery vehicle for targeting ASOs to Kupffer cells. (Ponnappa, B. C., et al., J. Liposome Res. 8: 521-535, 1998). Ninety minutes post intravenous injection, over 50% of liposome-encapsulated ASO was distributed in macrophage-rich organs, liver (40%) and spleen (10%), while incorporation into other organs such as muscle, heart, brain, lungs, kidneys and testes was minimal (Ponnappa, B. C., et al., J. Liposome Res. 8: 521-535, 1998). In the liver, over 65% of the ASO was found in Kupffer cells, accounting for a 200-fold enrichment of ASO in Kupffer cells versus that in the combined body tissues. (Ponnappa, B. C., et al., J. Liposome Res. 8: 521-535, 1998).

In addition to targeting primarily macrophages, a second challenge in the delivery of a therapeutic agent lies in the release of the therapeutic agent from the lysosomes. The anionic liposomal preparations used in previous studies (Ponnappa, B. C., et al., J. Liposome Res. 8: 521-535, 1998), although efficiently taken up by the macrophages, contained phosphatidyl choline, which renders the liposomes insensitive to the acidic pH (Ellens, H., et al, Biochemistry 23: 1532-1538, 1984), in the lysosomal vesicles. The pH-insensitivity makes them unsuitable for cytoplasmic delivery of a therapeutic agent, since, following phagocytosis, these liposomes remain trapped in the lysosomes until the contents are digested at acidic pHs by the powerful lysosomal hydrolases, possibly destroying the therapeutic agent. The initial studies with these pH-stable liposomal vesicles, which used the ASO TJU-2755 (SEQ. ID. NO: 1) as the therapeutic agent to target TNF-α mRNA, failed to show any therapeutic effect of the ASO (infra). Hence, it is necessary to develop alternative formulations of anionic liposomes, which at low pHs, will destabilize the endosomal/lysosomal barrier such that pharmacologically relevant concentrations of a therapuetic agent are released into the cytosol and nucleus. The formulation of the present invention uses phosphatidyl ethanolamine, which is fusogenic at acidic pHs (Ellens, H., et al., Biochemistry 23: 1532-1538, 1984) cholesteryl hemisuccinate which stabilizes the liposomes at physiologic pH, and cholesterol, a membrane stabilizing agent. Based on previous observations (Ellens, H., et al, Biochemistry 23: 1532-1538, 1984; Straubinger, R. M., Methods in Enzymology 221: 361-376, 1993; Connor, J., Huang, L., J. Cell Biol. 101: 582-589, 1985; Tschiakowsky, K., Brain, J. D., Shock 1: 401-407, 1994), it is expected that upon endocytosis and acidification by a proton pump in the membrane, pH-sensitive liposomes will fuse with the endosomal membrane and destabilize the endosomal compartment resulting in the release of the contents into the cytosol. The mechanism of delivery of macromolecules into the cytoplasm by pH-sensitive liposomes has been reviewed. (Straubinger, R. M., Methods in Enzymology 221: 361-376, 1993). Rapid entry of ASOs from the cytoplasm into the nucleus has been reported. (Fisher, T. L., et al., Nuclei Acid Res. 21: 3857-3854, 1993).

The present invention discloses herein the in vivo efficacy of the liposome formulation by using an anti-TNF-α ASO, TJU-2755 (SEQ. ID. NO: 1), as an example of a therapeutic agent that is delivered to the interior of a macrophage. Following encapsulation in pH-sensitive liposomes, in vivo delivery is accomplished by intravenous injection. Rats are subsequently administered LPS, following which plasma TNF-α levels and the ability of liver slices incubated ex vivo to produce TNF-α are determined. Results show that ASO TJU-2755 (SEQ. ID. NO: 1) effectively inhibits the ability of the liver to produce TNF-α and lowers plasma TNF-α levels, demonstrating a therapeutic potential of this delivery system in vivo. The use of ASO TJU-2755 (SEQ. ID. NO: 1) as a therapeutic agent delivered to macrophages is merely an example of a therapeutic agent that is incorporated into the liposome formulation of the present invention and is in no way meant to limit the use of the liposome. It will be obvious to those of ordinary skill in the art that variations in the therapeutic agent may be used and that it is intended that the invention may be practiced otherwise than as specifically described herein. Accordingly, this invention includes all modifications encompassed within the spirit of the invention defined by the claims.

### ABBREVIATIONS

“PE” means “phosphatidyl ethanolamine”

“CHEMS” means “cholesteryl hemisuccinate”

“DPPC” means “dipalmitoyl phosphatidy choline”

“DPPG” means “dipalmitoyl phosphatidy glycerol”

“ASO” means “antisense oligonucleotide”

“TNF-α” means “tumor necrosis factor-alpha”

“LPS” means “endotoxin or bacterial lipopolysaccharide”

### SUMMARY OF THE INVENTION

The present invention provides a composition of matter, wherein a liposome has a lipid component including a phosphatidyl ethanolamine, a cholesteryl hemisuccinate, and cholesterol component. The phosphatidyl ethanolamine component renders the liposome fusogenic at acidic pHs and the cholesteryl hemisuccinate component, together with the cholesterol component, renders the liposome stable at physiological pHs. In one embodiment the composition of matter has phosphatidyl ethanolamine, cholesteryl hemisuccinate, and cholesterol in a molar ratio of 7:4:2. In another embodiment the acidic pHs comprise a pH between 5 and 6. In a further embodiment the physiological pHs are a pH between 7 and 8. In another embodiment the liposome has a diameter between 0.2-2.0μ.

It is an object of the present invention to provide a pharmaceutical composition that includes a pharmaceutically acceptable carrier and a liposome, wherein the liposome has a lipid component including a phosphatidyl ethanolamine, a cholesteryl hemisuccinate, and cholesterol component.

It is another object of the present invention to provide a method of treating a macrophage associated disease or condition in a mammal. A therapeutically active agent is encapsulated into a liposome having lipid components which include a phosphatidyl ethanolamine, a cholesteryl hemisuccinate, and a cholesterol. The phosphatidyl ethanolamine component renders the liposome fusogenic at acidic pHs and the cholesteryl hemisuccinate component, together with the cholesterol component, renders the liposome stable at physiological pH. The liposome is administered to the mammal, thereby delivering the liposome to a macrophage. The liposome is taken up by the macrophage, and subsequently fuses with a lysosome in the macrophage, thereby destabilizing the liposome fused with the lysosome in the macrophage, and releasing the therapeutic agent into the cytosol of the macrophage.

### DESCRIPTION OF THE FIGURES

FIG. 1. Stability of pH-sensitive liposomes. Liposomes encapsulated with trace amounts of labeled (³²P) ASO TJU-2755 (SEQ. ID. NO: 1) plus 100 μg of unlabeled TJU-2755 (SEQ. ID. NO: 1) are prepared by the reverse phase method (infra). Aliquots of the liposomal suspension are exposed to varying pHs (pH 5.0-7.4) in PBS for 15 min at 37° C. and centrifuged at 100,000× g for 45 min. The amount of radioactivity (ASO) remaining in the liposomes is expressed as percent of the total radioactivity present in the suspension at the beginning of the experiment, at pH 7.4. Values are the means ±S.E. of n=4 separate preparations.

FIG. 2. Long-term stability of pH-sensitive liposomes. Liposomes are prepared in the presence of 2 mg of ASO TJU-2755 (SEQ. ID. NO: 1) plus trace amounts of (³²P) TJU-2755 (infra). The liposomal preparation is divided into several aliquots and stored at 4° C. At the times indicated, aliquots of the suspension are centrifuged at 100,000× g to separate the supernatant from the pellet. The amount of radioactivity associated with the pellet is expressed as percent of that present in the pellet on the day of the preparation (day “zero”). Values are the means of two separate preparations, each of which prepared on different days.

FIG. 3. Stability of liposomes in plasma. Liposomes containing trace amounts of labeled (³²P) ASO TJU-2755 (SEQ. ID. NO: 1) are prepared as described in legend to FIG. 1. Aliquots of the liposomal preparation are mixed with rat plasma (10% dilution of plasma) and incubated at 37° C. for 60 min. The final concentration of liposomal lipid ranges from 0-4 mg/ml plasma. After incubation, the suspensions are diluted with PBS and ultracentrifuged to separate the supernatant from the liposomes. The data points are the means of two closely agreeing values obtained from separate liposomal preparations.

FIG. 4. In vivo efficacy of ASO TJU-2755 (SEQ. ID. NO: 1) against TNF-α in the liver: Effect of duration post-injection. Rats are intravenously injected with pH-sensitive liposomes encapsulated with TJU-2755 (1.5-1.75 mg/Kg body wt). At different times (24, 48 and 72 h) after ASO administration, LPS (50 μg/Kg body weight for 90 minutes) is administered intravenously (i.v.) and the animals are sacrificed 90 min after this challenge. Liver is excised, slices prepared and incubated for 2 h (infra). The amount of TNF-α released into the medium is measured by ELISA. The TNF-α values (bars) from the ASO-treated animals are normalized to control values (control=100) obtained from body weight-matched rats treated similarly except that they are injected with “empty” liposomes. Values indicated are means ±S.E. of (n) determinations. Number of animals, 24 h, n=3; 48 h, n=4, 72 h, n=3. * p<0.05 (vs Control).

FIG. 5. In vivo efficacy of ASO TJU-2755 (SEQ. ID. NO: 1) against TNF-α in the liver: Effect of multiple injections. Rats are injected with either one or two daily doses of ASO (1.5-1.75 mg/Kg body wt) encapsulated liposomes. Forty eight hours after the last injection, they are administered LPS (50 μg/Kg) and the animals are sacrificed 90 min after this challenge. The amount of TNF-α produced by liver slices is determined as described in legend to FIG. 4 (supra). Bars indicate the amount of TNF-α produced by ASO-treated animals, expressed as percent of controls injected with “empty” liposomes and are the means ±S.E. of n=4 for single dose and n=7 for two doses. *p<0.05, **p<0.01 vs control). The absolute values (Mean) of TNF-α for control and TNF-2755-treated livers (for two daily doses) were 17,748 and 7,500 pg/g liver, respectively.

FIG. 6. Effect of multiple doses of ASO TJU-2755 (SEQ. ID. NO: 1) on plasma TNF-α. Rats are injected with either one (n=3), two (n=8) or three (n=1) daily doses of TJU-2755 (SEQ. ID. NO: 1) and administered LPS (50 μg/Kg body wt.) at 48 h after the last injection of the antisense formulation as described in legend to FIG. 5. Ninety minutes after the LPS injection, rats are sacrificed and blood is collected in heparinized tubes. Plasma TNF-α is determined by ELISA. Bars indicate values from ASO-treated animals expressed as percent of corresponding controls (animals dosed with “empty liposomes”) and are the means ±S.E of (n) determinations. ***p<0.001 between rats injected two daily doses of ASO and controls. The absolute (Mean) values of TNF-α for control and TJU-2755 (SEQ. ID. NO: 1)-treated plasma samples (for two daily doses) were 34,375 and 9,731 (pg/ml plasma) respectively.

FIG. 7. Effect of multiple doses of ASO TJU-2755 (SEQ. ID. NO: 1) on tissue levels of the oligonucleotide. Rats are injected with either one (n=4), two (n=5) or three (n=1) daily doses of liposome-encapsulated ASO. Forty eight hours after the last injection, they are administered LPS (50 μg/Kg body wt.). Ninety minutes after the LPS injection, rats are sacrificed and a portion of the liver is processed for the extraction and estimation of the ASO (infra).

FIG. 8. Effect of ASO TJU-2755 (SEQ. ID. NO: 1) in vivo on TNF-α mRNA. Rats are injected with two daily doses (1.5 mg/Kg body wt.) of ASO and injected with LPS (50 μg/Kg body wt.) 48 h post ASO administration as described in legend to FIG. 5. Liver (n=5) and spleen (n=4) are processed for the extraction of mRNA fractions. Steady-state levels of TNF-α mRNA are determined by Northern hybridization and normalized to the levels of GAPDH mRNA. Bars indicate mRNA (Means ±S.E) of (n) values from ASO-treated samples, expressed as percent of control values derived from rats treated with “empty” liposomes. *p<0.05, **p<0.01 (Vs control).

### DESCRIPTION OF THE INVENTION

Materials and Methods

Animals:

All animal experiments are carried out in male Sprague Dawley rats purchased from Harlan Sprague Dawley Inc., Indianapolis, Ind. Animals are maintained on laboratory-chow. The body weights of the animals ranged from 250-300 g. The experimental protocol is approved by the Institutional Animal Care and Use Committee of Thomas Jefferson University, Philadelphia.

Chemicals:

Cholesterol, cholesteryl hemisuccinate (CHEMS), dipalmitoyl phosphatidy choline (DPPC) and dipalmitoyl phosphatidy glycerol (DPPG) are purchased from Sigma Chemicals Co., St. Louis, Mo. Phosphatidyl ethanolamine (PE) (transphosphatidylated from egg lecithin) is purchased from Avanti Polar Lipids Inc., (Alabaster, Ala.). Radioisotope [γ-³²P]-ATP, is purchased from Amersham Corp (Arlington Heights, Ill.). The phosphorothioate oligonucleotides used are custom synthesized either from Geneset Inc (La Jolla, Calif.), or from Hybridon Inc., (Milford, Mass.). Essentially two types of oligonucleotides are used in the present invention and both are 21 nucleotides long. The first one, ASO TJU-2755 (SEQ. ID. NO: 1), had a sequence (5′-TGATCCACTCCCCCCTCCACT-3′; SEQ. ID. NO: 1), complementary to the 3′-untranslated region of rat TNF-α. mRNA. (Tu et al., 1998). The second oligo, TJU-2755SS (SEQ. ID. NO: 2), was a “sense” oligonucleotide, complementary to TJU-2755 (SEQ. ID. NO: 1) (Tu, G-C., et al., J. Biol. Chem. 273: 25125-25131, 1998). All other chemicals used are of reagent grade, purchased either from Sigma Chemicals Co. or from Fisher Scientific, Pittsburgh, Pa.

Preparation of Labeled Phosphorothioate ASO:

Where indicated, the ³²P-ASO used in this study is labeled at the 5′-end with [γ-³²P]-ATP using a DNA 5′-end labeling System kit from Promega (Madison, Wis.). Routinely, two hundred nanograms of the oligo is incubated with [γ-³²P]-ATP (3000-5000 Ci/mmole) and T4-polynucleotide kinase at 37° C. for 30 min. The reaction is stopped by the addition of EDTA to a final concentration of 0.1M and precipitated with ethanol overnight at −20° C. The precipitate is sedimented by centrifugation, washed twice with 80% ice-cold ethanol, dried and dissolved in TE buffer (10 mM Tris and 1 mM EDTA, pH 8.0).

Preparation of ASO-Encapsulated pH-Sensitive Liposomes:

An amphipathic lipid such as PE can form a stable bilayer only at pH greater than 9 or in media of low ionic strength. Consequently, the bilayer collapses in a physiologcal (pH) environment. A secondary amphipath such as CHEMS provides a conditional stability, such that the liposomes are stable at pH 7-8 but collapse in acidic environment (pH<6).

Liposomes encapsulated with ASO are prepared by the reverse phase method originally described by Szoka and Papahadjopolous (1978) with slight modifications. Briefly, a mixture of PE, CHEMS and cholesterol (molar ratio of 7:4:2) is dissolved in chloroform. Routinely, 20-25 mg of TJU-2755 (SEQ. ID. NO: 1) is dissolved in 0.4 ml of TE buffer and diluted to 1.5 ml with ten-fold diluted PBS (phosphate buffered saline). The ASO solution is mixed with 3 volumes of chloroform (containing 25 mg of lipid mixture) and sonicated in a bath-type sonicator for 5 min. The organic solvent is evaporated at ambient temperature using a rotary-type evaporator. The resultant liposomal suspension is diluted with buffer (1/10 PBS) and centrifuged at 100,000× g for 45 min to separate the liposomes from the medium. The pellet is washed twice with PBS and finally resuspended in a volume not exceeding 1 ml of PBS. To determine the amount of encapsulated oligonucleotide, an aliquot of the liposomal preparation is treated with a mixture of chloroform and methanol (1:1 v/v) to release the contents. The solvent is evaporated and the oligonucleotide is extracted in TE buffer. The amount of ASO in solution is quantitated spectrophotometrically or by Southern hybridization (infra). Depending upon the concentration of ASO used, the encapsulation efficiency varies from 15-75%. Routinely, for 20-25 mg of ASO, the encapsulation efficiency ranges from 17-20%.

pH-Sensitivity of Liposomes:

Liposomes are prepared (supra) encapsulating trace amounts of (³²P)-TJU-2755 (SEQ. ID. NO: 1) (SEQ. ID. NO: 1) plus 100 μg of the unlabeled ASO. Aliquots of the liposomal suspension are exposed to varying pH conditions, in the range of pH 5-7.4 at 37° C. for 15 min. The suspensions are centrifuged at 100,000× g for 45 min at 4° C. to separate the supernatant from the liposomes. The amount of radioactivity released into the supernatant is expressed as percent of the radioactivity that is present in the total suspension.

Liposomal Storage Stability and Size Determination Studies:

To determine the stability of the liposomes upon storage, trace amounts of TJU-2755 (SEQ. ID. NO: 1) are labeled with [γ-³²P] ATP (supra) and mixed with 2 mg of the unlabeled ASO. Liposomes are prepared (supra) and stored at 0-4° C. At various intervals up to 4 weeks, aliquots of the liposomal suspension are centrifuged at 100,000× g for 45 min to separate the liposomes from the medium. The amount of radioactivity retained in the liposomes is compared with that present on the day of preparation, “day zero”.

The mean liposome diameter is determined by Quasi-elastic light scattering using Coulter N4MD, Hialeah, Fla. An Unimodal (cumulants) fit to log-Gaussian distribution is applied to determine the mean particle size (Uster, P. S., et al., FEBS Letters 386: 243-246, 1996). The measurements are carried out at 25° C.±0.5° C. Each sample contained 150 μg (dry lipid weight) in 3.5 ml PBS, pH 7.4.

Stability of pH-Sensitive Liposomes in Plasma:

Plasma is obtained from heparinized rat blood. Liposomes containing labeled TJU-2755 (SEQ. ID. NO: 1) are prepared (supra) for the determination of pH stability. Aliquots of the liposomes are mixed with rat plasma and incubated at 37° C. to a final concentration range of 0-4 mg lipid/ml. The final concentration of plasma is at least 90%. In the control group, plasma is replaced by PBS. The mixture is incubated at 37° C. for 5, 30 and 60 min. At the end of incubation, the contents are diluted 10-fold with PBS and ultracentrifuged to separate the supernatant from the liposomes. The amount of radioactivity associated with the pellet as well as the supernatant are determined. The amount of radioactivity in the supernatant is expressed as a percent of the total amount of radioactivity that is present in the original liposomal suspension.

Preparation of pH-Stable Liposomes:

pH-stable liposomes are prepared by the reverse phase method exactly as described (supra) for pH-sensitive liposomes except that a mixture of DPPC, DPPG and cholesterol (molar ratio of 4:1:5) is substituted for the lipids. Routinely, 15 mg of ASO and 25 mg of the lipid mixture are taken for the preparation of pH-stable liposomes. The encapsulation efficiency ranged from 16-20%.

In Vivo Studies:

Liposomes encapsulated with TJU-2755 (SEQ. ID. NO: 1) are injected intravenously (in a volume of PBS not exceeding 1 ml) into the animal by the tail vein route. In all of the experiments, the concentration of the liposomal lipid injected is maintained in the range of 15-17 mg/Kg body weight. Because the encapsulation efficiency varied from preparation to preparation, the amount of TJU-2755 (SEQ. ID. NO: 1) injected also varied. However, unless indicated otherwise, the amount of TJU-2755 (SEQ. ID. NO: l) injected ranged between 1.5-2.0 mg/kg body weight. Rats are injected single or multiple daily doses of the ASO, and sacrificed 24, 48 or 72 h post injection, as per specific experiments detailed in figures legends. Ninety minutes prior to sacrifice, animals are administered LPS (50 μg/kg body weight). Venous blood is collected in a heparinized tube 90 min post-injection and is kept on ice until processed for TNF-α. Liver, and when necessary, spleen are taken for processing (infra).

TNF-α Secretion in the Liver:

Following the in vivo administration of LPS, the production of TNF-α in the liver is assessed by measuring the amount of TNF-α secreted by liver slices. Liver slices are prepared and incubated in culture medium according to the procedure described by Videla and Israel (Videla, L., Israel, Y., Biochem J. 118: 275-281, 1970) with modifications, which include the addition of insulin and fetal calf serum to the culture medium. Briefly, liver slices of uniform thickness (10×4×0.4 mm) are prepared from the mid lobe and initially rinsed in ice-cold William's E medium. The slices (2 slices per dish) are transferred to culture dishes (35 mm) containing 2 ml of fresh medium (90% William's E, 10% fetal calf serum and 2 units of insulin/100 ml) and incubated at 37° C. for 2 h. The amount of TNF-α released into the medium is measured (infra).

Enzyme-Linked Immunoabsorbent Assay (ELISA) of TNF-α:

ELISA is conducted by using Cytoscreen KRC3012 kits (Biosource, Camarillo, Calif.) according to the manufacturer's specifications. Supernatants containing high TNF-α levels are diluted prior to the assay to assure assay results within the standard curve.

Assay of ASO by Southern Hybridization:

Extraction:

The ASO TJU-2755 (SEQ. ID. NO: 1), is extracted from the tissues and quantitated by Southern hybridization. (Ponnappa, B. C., et al., J. Liposome Res. 8: 521-535, 1998). About 100 mg of the wet tissue is homogenized in an ice-cold buffer (50 mM Tris, pH 7.5, 10 mM EDTA), hereafter referred to as the homogenization buffer. Five-fold and twenty-fold volumes of the homogenizing buffer are used for liver and spleen tissues respectively. Subsequent digestion and extraction of the oligonucleotide is carried out according to the procedure of Temsamani (Temsamani, J., et al., Analytical Biochem 215: 54-58, 1993) with modifications. Briefly, 100 μl of the homogenate is mixed with proteinase K (Sigma Chemicals Co.) solution (3.6 mg/ml) to a final volume of 150 μl. To this, an equal volume of an extraction buffer (0.5% SDS, 10 mM NaCl, 20 mM Tris-HCl (pH 7.6), 10 mM EDTA) is added and incubated at 55° C. for 2 h. The digest (0.3 ml) is then mixed with an equal volume of the extraction solvent (phenol: chloroform:isoamyl alcohol (25:24:1 v/v; GIBCO BRL, Gaithesberg, Md.), vortexed vigorously and centrifuged in a microfuge for 2 min. The aqueous upper phase is transferred to a fresh tube and the organic lower phase is re-extracted once with 100 μl of TE buffer. The aqueous supernatants are combined and further extracted with an equal volume of chloroform. The ASO is precipitated (−20° C./overnight) from the final aqueous extract by the addition of ethanol (70% v/v final) in the presence of 0.3 M sodium acetate (pH 5.3). The precipitate is sedimented by centrifugation, washed once with ice-cold 80% ethanol, dried and dissolved in 100 μl of TE buffer.

Quantitation:

The ASO TJU-2755, (SEQ. ID. NO; 1) is separated by PAGE (20% acrylamide/7M urea), transferred to a nylon membrane (Hybond) and cross-linked (Stratalinker). The membrane is pre-hybridized in a sealed bag for 1 h at 40° C. using Expresshyb Hybridization Solution® (Clonetech, Palo Alto, Calif. ) and hybridized for 3 h in fresh buffer containing labeled (P-32) probe, a 21-mer oligonucleotide TJU-2755SS (SEQ. ID. NO: 2) complementary to TJU-2755 (SEQ. ID. NO: 1). Routinely, the isotope concentration in the hybridization buffer is 2-3×10⁶ cpm/ml. Following hybridization, the membrane is washed in 5× SSC (10 min) and 5× SSC/0.05% SDS (5 min) at 40° C. The blotted membrane is placed between the folds of a Saran wrap and exposed to X-ray film overnight at −70° C. The autoradiograms are scanned (Model JX-330, Sharp Corporation, Japan, equipped with Image Scan software) and the densities associated with bands (contours) are compared. In each gel, along with the samples, varying concentrations of the standard TJU-2755 (SEQ. ID. NO: 1) (2.5 to 20 ng) are also included. The concentration of the unknown is extrapolated from the standard curve.

Tissue Levels of TNF-α mRNA by Northern Hybridization:

Extraction of RNA:

Liver and spleen samples (≈100 mg) are homogenized in 3 volumes of cold TRI Reagent (Molecular Research Center, Inc.). The resultant homogenate is treated with chloroform for phase separation, precipitated with isopropanol, and washed with ethanol (70% v/v). The purified RNA pellet is resuspended in diethylpyrocarbonate (DEPC)-treated water and kept frozen until further analysis. The intactness of total RNA is measured by agarose (1%) gel electrophoresis by visualizing the intactness of ribosomal RNA (28S, 18S). Isolation of mRNA from total RNA is accomplished using Qiagen's Oligotex mRNA Spin-Column Midi Kit as per protocol. The purity of the isolated mRNA is monitored by analyzing the absorption ratios of 260 to 280 (nm), which always ranged from 1.8-2.0.

Quantitation of mRNA:

Samples of mRNA are run on 1% agarose gel electrophoresis, vacuum transferred (BIORAD Model 785 Vacuum Blotter) onto a nitrocellulose membrane (Amersham), and crosslinked (Statagene UV Stratalinker 2400). Subsequently, the membrane is prehybridized overnight at 50° C. (PerfectHyb Plus Hybridization Buffer, Sigma). The next day, the DNA probe (322 bp PCR fragment from the open reading frame of rat TNF-α gene) is labeled with ³²P using a random labeling system (Amersham Multiprime DNA Labeling System RPN.1601). Hybridization is carried out overnight at 50° C. in fresh hybridization solution plus the labeled DNA probe (specific activity≈10⁹ cpm/μg). The membrane is washed at 55° C., sequentially in 4× SSC, 2× SSC, 1× SSC/0.1% SDS, 0.2× SSC/0.1% SDS, and 0.1× SSC/1% SDS. The membrane is briefly air-dried and exposed to X-ray film to obtain the autoradiograms. The autoradiograms are quantitated using an ultrascan laser densitometer. The membrane is then stripped and rehybridized with cDNA probe for rat GAPDH (glyceraldehyde phosphate dehydrogenase) used as the housekeeping gene. The intensities of the TNF-α mRNA bands are normalized to values obtained with the housekeeping gene.

Results

Size, pH Sensitivity and Long-Term Stability of pH-Sensitive Liposomes

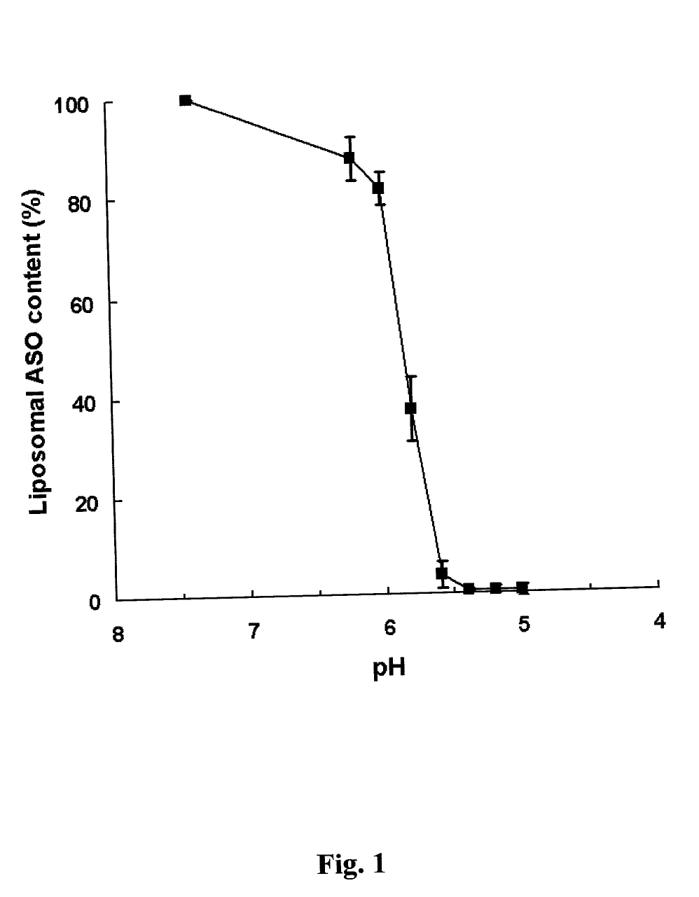

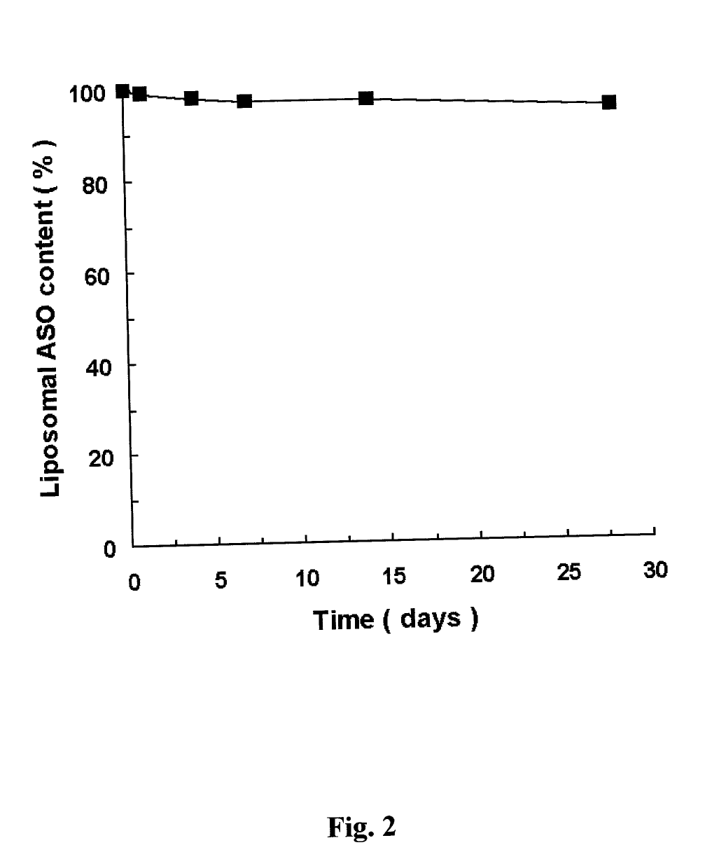

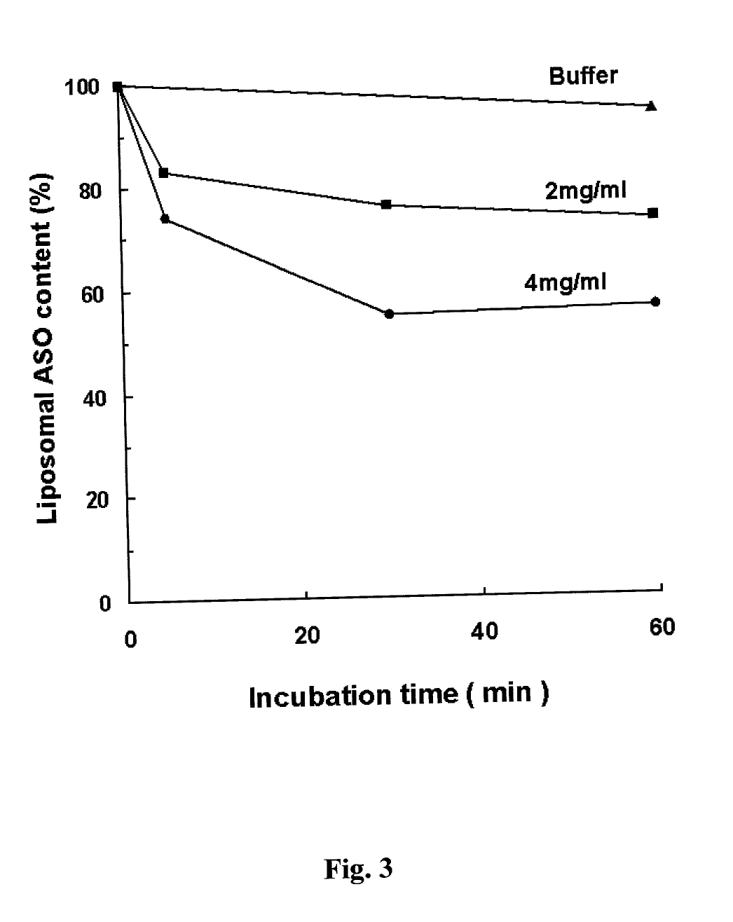

In a typical preparation, 85% of the liposomes are 0.2-1.0 μ in diameter; 5% were <0.2μ, and 10% are >1 μ. The liposomes are stable at pH 7.4. As expected, when the pH is lowered, the liposomes become unstable, losing about 20% of the oligonucleotide content at pH 6 and 100% at pH 5.5 (FIG. 1). The liposomes display great stability at pH 7.4 at 4° C.; when stored under these conditions for 4 weeks, over 95% of the ASO remains encapsulated in the liposomes (FIG. 2). The stability of liposomes in plasma is also tested. As shown in FIG. 3, the stability of liposomes is dependent upon the concentration of lipid in plasma: the higher the concentration, the greater the instability. At 2 mg lipid/ml of plasma, >80% of the liposomes remains intact after 60 min incubation at 37° C. Since the concentration of liposomes attained in vivo after intravenous injection is in a much lower range (0.5-0.6 mg lipid/ml plasma), they remain intact during the period of rapid sequestration by the macrophages, similar to that reported for other anionic liposomes. (Ponnappa, B. C., et al., J. Liposome Res. 8: 521-535, 1998).

In vivo Efficacy of ASO TJU-2755 (SEQ. ID. NO: 1)

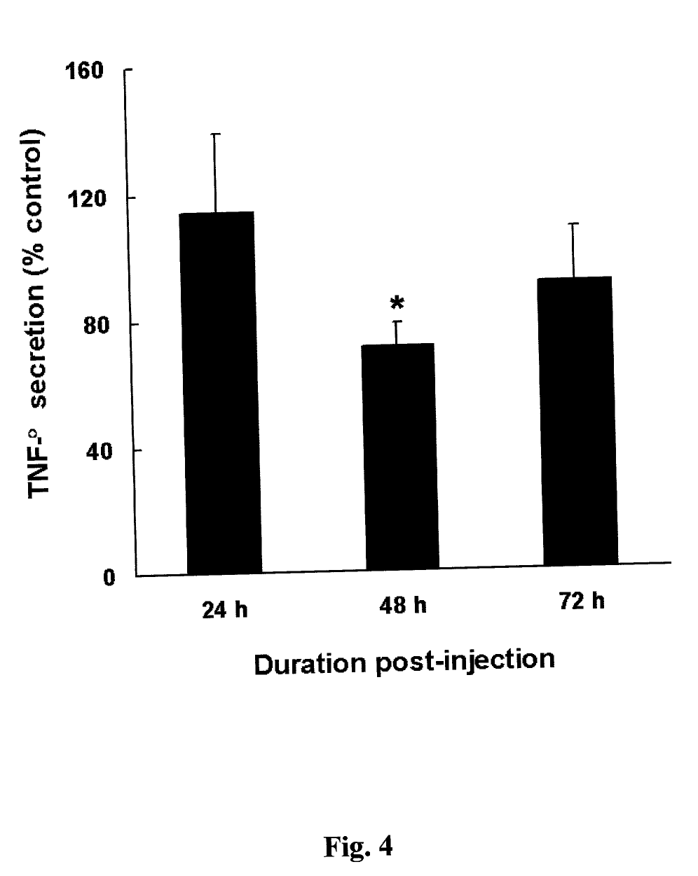

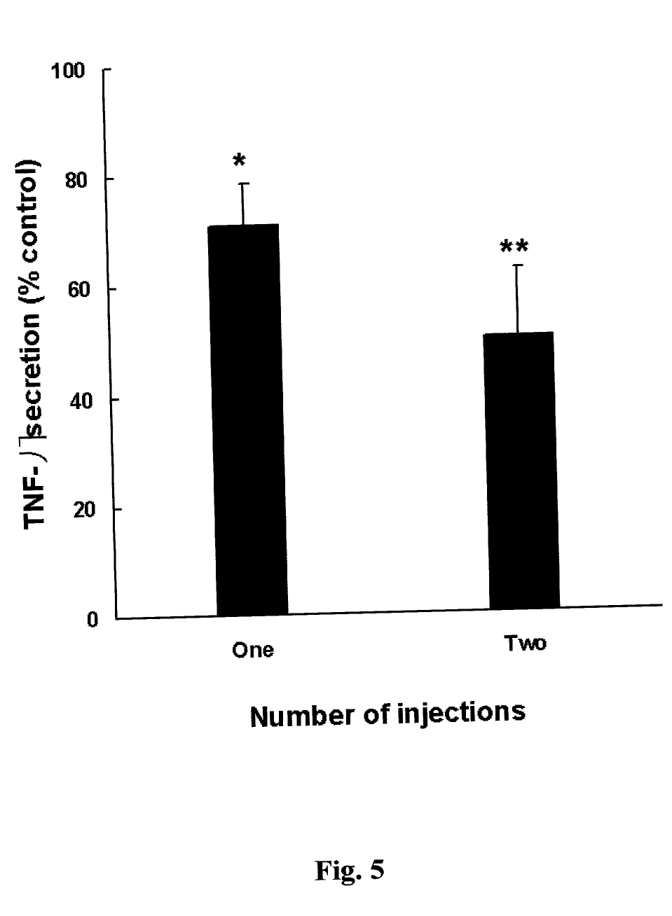

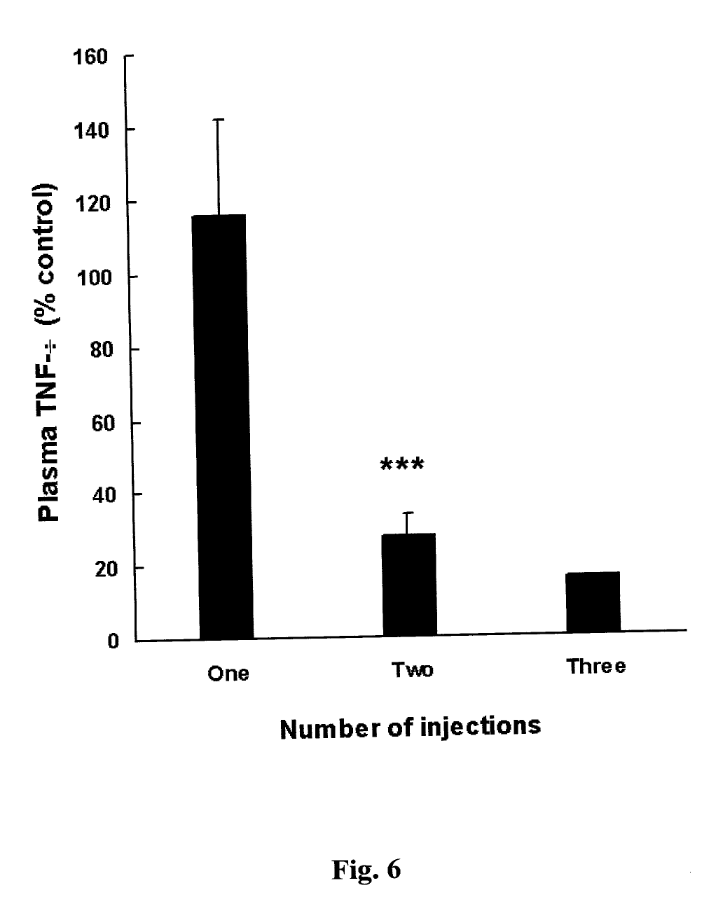

Delivery of liposome contents is readily assessed by ordinary skilled artisans given the teachings of this invention, using routine techniques. The efficacy of TJU-2755 (SEQ. ID. NO: 1), administered in vivo, is assessed by two different methods. One measures the amount of TNF-α produced by the liver tissue itself and the other determines plasma TNF-α levels. In both cases, the animals are administered LPS prior to determination of TNF-α. Based on preliminary observations, it is expected that there will be a time lag between the phagocytosis of liposomes by the macrophages and the entry of the oligonucleotides into the nucleus. In the initial studies, the effect of a single intravenous administration of ASO TJU-2755 (SEQ. ID. NO: 1) on LPS-induced production of TNF-α is determined in liver slices. The studies show that maximal efficacy (˜30% inhibition) is observed when LPS is given 48 h post injection (FIG. 4). Therefore, in subsequent studies, the effect of multiple daily injections of TJU-2755 (SEQ. ID. NO: 1) is tested 48 h after the last LPS injection. As shown in FIG. 5, the extent of inhibition increases with the number of daily doses, reaching 50% after two daily doses (p<0.01). A more pronounced reduction (68%, p<0.001, n=8) in TNF-α levels is observed in the corresponding plasma samples following 2 doses of TJU-2755 (SEQ. ID. NO: 1) (FIG. 6). The plasma TNF-α values (LPS treated) for rats administered “empty” liposomes at 15-17 mg lipid/Kg body weight are not significantly different from those for PBS injected controls.

In contrast to the inhibitory effects of TJU-2755 (SEQ. ID. NO: 1) using pH-sensitive liposomes, when the ASO is delivered with pH-stable liposomes, no inhibitory effect on LPS-induced TNF-α production is observed (Table 1).

TABLE I
pH-stable liposomes and antisense action

| | Control | ASO 2755 | |
|---|---|---|---|
| Sample | TNF-α | (%) | n = |
| Liver | 100 ± 17 | 103 ± 23 | 8 |
| Plasma | 100 ± 15 | 93 ± 28 | 7 |

Rats are injected intravenously with two daily doses (1.5 mg/Kg body wt.) of ASO TJU-2755 (SEQ. ID. NO: 1) encapsulated in pH-stable liposomes and challenged with LPS (50 μg/Kg body wt.) at 48 h post injection. Animals are sacrificed 90 minutes post-LPS. The amount of TNF-α in plasma, as well as that secreted by liver slices, is determined exactly as described for pH-sensitive liposomes (supra) and legends to FIG. 4 and FIG. 5. Body-weight matched rats injected with two doses of “empty” liposomes and challenged similarly with LPS constituted the control group. All TNF-α values are normalized to the corresponding control groups (=100). Values are expressed as mean ±S.E of (n) determinations.

Tissue Levels of ASO TJU-2755 (SEQ. ID. NO: 1)

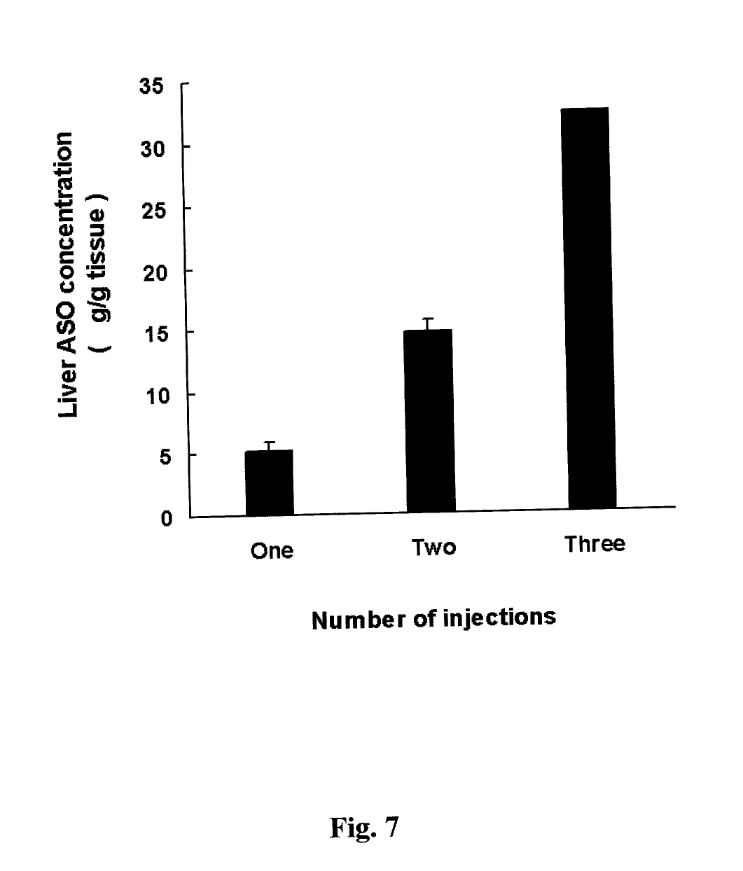

Multiple doses of the ASO have a cumulative effect on tissue levels of intact TJU-2755 (SEQ. ID. NO: 1) as measured by Southern hybridization. As shown in FIG. 7, using pH-sensitive liposomes, at 48 h post injection, a single dose (1.75 mg/kg body wt.) of TJU-2755 (SEQ. ID. NO; 1) results in a concentration of 5.8 μg/g tissue of liver. Under similar conditions, two daily doses of the ASO led to an accumulation of 14.7 μg/g tissue and three daily doses have an accumulation of 32.3 μg/g tissue.

TNF-α mRNA Levels Following the Administration of ASO TJU-2755 (SEQ. ID. NO: 1)

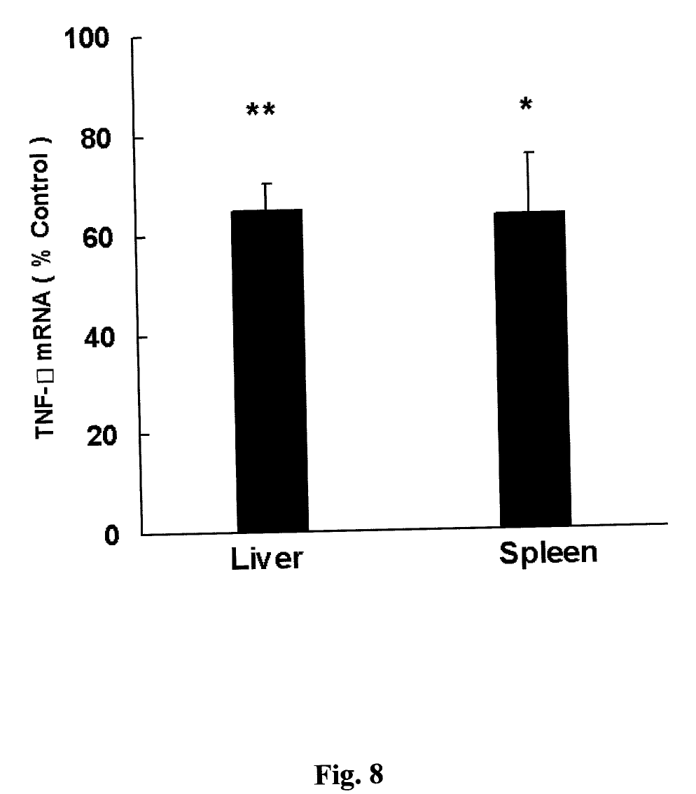

Since, it has been reported that binding of ASOs to the target mRNA transcripts initiated RNase H-mediated degradation of mRNA (Giles et al., 1995), the inhibitory effects of TJU-2755 (SEQ. ID. NO: 1) on LPS-induced TNF-α production in viv, could also be due to reduced levels of TNF-α mRNA, as reported earlier in isolated Kupffer cells. (Tu, G-C, et al, J. Biol. Chem. 273: 54-58, 1998). As shown in FIG. 8, in the liver tissue, there is a 35% reduction (p<0.01) in the steady-state levels TNF-α mRNA in the group pre-treated with TJU-2755 (SEQ. ID. NO: 1) as compared to controls (“empty” liposomes). It is reasonable to assume that LPS-induced changes in TNF-α mRNA in the whole liver tissue reflects, to a large extent but not exclusively, effects on Kupffer cells. Similar to the liver, the spleen also contains of macrophages which are targeted by anionic liposomes. (Ponnappa, B. C., et al., J. Liposome Res. 8: 521-535, 1998). For comparative purposes, TNF-α mRNA is determined in the spleen following LPS administration, and is found to be similarly affected by TJU-2755 (SEQ. ID. NO: 1), as shown by a 36.5% reduction (p<0.05) in mRNA levels (FIG. 8).

Therapeutic Compositions

The method of the present invention can be used in one of many different mammalian species, including but not limited to, bovine, ovine, porcine, equine, rodent and human. Further provided herein is a method of delivering a therapeutic agent to a cell. “Therapeutic agents” which may be delivered by the liposomes into cells are any compound or composition of matter that can be encapsulated in liposomes and administered to a mammal, preferably humans. Liposomes can be loaded with therapeutic agents by solubilizing the agent in the lipid or aqueous phase used to prepare the vesicles. Alternatively, ionizable therapeutic agents can be loaded into liposomes by first forming the liposomes, establishing an electrochemical potential, e.g., by way of a pH gradient, across the outermost liposomal bilayer, and then adding the ionizable agent to the aqueous medium external to the liposome (see Bally et al. U.S. Pat. No. 5,077,056, the contents of which are incorporated herein by reference).

Therapeutic agents can have therapeutic activity in mammals, and can also be administered for diagnostic purposes. Therapeutic agents which may be associated with this invention's liposome include, but are not limited to: antiviral agents such as acyclovir, zidovudine and the interferons; antibacterial agents such as aminoglycosides, cephalosporins and tetracyclines; antifungal agents such as polyene antibiotics, imidazoles and triazoles; antimetabolic agents such as folic acid, and purine and pyrimidine analogs; antineoplastic agents such as the anthracycline antibiotics and plant alkaloids; carbohydrates, e.g., sugars and starches; amino acids, peptides, proteins such as cell receptor proteins, immunoglobulins, enzymes, hormones, neurotransmitters and glycoproteins; dyes; radiolabels such as radioisotopes and radioisotope-labeled compounds; radiopaque compounds; fluorescent compounds; mydriatic compounds; bronchodilators; nucleic acid sequences such as messenger RNA, cDNA, genomic DNA and plasmids; anti-inflammatory agents, such as curcumin and the like.

Delivery can occur in vitro, such as for diagnostic purposes. Preferrably, the contacting is in vivo, in which case the cells are preferably mammalian, and a pharmaceutically acceptable carrier (infra) is used to deliver the liposomes of the present invention to a mammal. In vivo liposomal therapeutic agent delivery, according to the practice of this invention, will deliver therapeutically or diagnostically effective amounts of therapeutic or diagnostic agents into the cells of a mammal afflicted with a macrophage associated disease, disorder or condition amenable to diagnosis or treatment with the agent. Hence, such delivery fusion is used to diagnose or treat the mammal for the disease, disorder or condition. For example, the mammal can be afflicted with a septic condition, an infectious microbial disease, e.g., a viral or bacterial infection, or inflammatory condition, e.g., an arthritic condition or autoimmune disorder such as rheumatoid arthritis or juvenile diabetes, and a therapeutically effective amount of an antimicrobial or anti-inflammatory agent is delivered to the mammal's cells.

The delivery of liposomal contents to cells is facilitated by the incorporation of phosphatidyl ethanolamine (PE) into the liposomes, as the PEs destabilize the liposomes' bilayers in the presence of an acidic pH, as occurs intracellularly in the lysosomes. PE-mediated destabilization of endosomal/lysosomal membranes results in direct delivery of liposomal contents into the cells.

Pharmaceutical Compositions

Also provided herein are compositions containing the liposomes of this invention. Included in such compositions are pharmaceutical compositions that also comprise a “pharmaceutically acceptable carrier,” which is a medium generally acceptable for use in connection with the administration of liposomes to mammals, including humans. Pharmaceutically acceptable carriers are formulated according to a number of factors well within the knowledge of the ordinarily skilled artisan to determine and account for, including without limitation: the particular therapeutic agent used, its concentration, stability and intended bioavailability; the disease, disorder or condition being treated with the composition; the subject, its age, size and general condition; and the composition's intended route of administration. Pharmaceutically acceptable carriers can contain additional ingredients, such as those which enhance the stability of the liposomes.

Administration

The method of delivery is by the intravenous route. The liposomes may be administered, for example, by infusion or bolus injection, and may be administered together with other biologically active agents.

It may be desirable to administer the pharmaceutical compositions of the invention intravenously as a prophylactic, for example (but not limited to), against sepsis prior to surgery.

The amount of the encapsulated drug in the liposome of the invention that will be effective in the treatment of a particular disorder or condition will depend on the nature of the disorder or condition, and is determined by standard clinical techniques. In addition, in vitro assays may optionally be employed to help identify optimal dosage ranges. The precise dose to be employed in the formulation also will depend on the route of administration, and the seriousness of the disease or disorder, and will be decided according to the judgment of the practitioner and each patient's circumstances. Effective doses may be extrapolated from dose-response curves derived from in vitro or animal model test systems.

Discussion

The efficacy of the liposomal composition of the present invention is assessed by analyzing the in vivo efficacy of these liposomes to deliver antisense oligonucleotides to Kupffer cells, thereby suppressing the LPS-induced production of TNF-α. The efficacy of any antisense molecule depends upon its ability to cross the lysosomal barrier and be released into the cytoplasm. Cationic liposomes have been successfully used for DNA transfection in cell culture systems. However, because of their almost complete instability in undiluted plasma or sera (typically, in in vitro studies, serum is diluted 5-20%), their usefulness for in vivo delivery is limited.

pH-stable anionic liposomes have been used as an efficient delivery vehicle for targeting oligonulceotides to Kupffer cells in vivo (Ponnappa, B. C., et al., J. Liposome Res. 8: 521-535, 1998); however, the liposomes used in that study do not contain the pH-sensitive fusogenic lipid, phosphatidyl ethanolamine, and therefore, do not possess the ability to destabilize the endosomal/lysosomal membrane barrier. As shown in Table I, ASO TJU-2755 (SEQ. ID. NO: 1) is ineffective when delivered in that fashion. The liposomal composition of the present invention overcomes this barrier by incorporating the pH-sensitive fusogenic lipid phosphatidyl ethanolamine, as well as cholesterol hemisuccinate to maintain stability at physiological pH, into the liposomal lipid composition, thereby fulfilling a long sought, yet unfulfilled, need to effectively deliver compounds to the interior of a cell.

Macrophages contain scavenger receptors that recognize an array of negative charges such as in anionic liposomes, thereby allowing for the efficient uptake of the liposomes of the present invention. (Ponnappa, B. C., et al., J. Liposome Res. 8: 521-535, 1998; Bautista, A. P., et al., J. Leukocyte Biol. 55: 321-327, 1994). Sequestration by macrophages, of which the Kupffer cells in the liver are an example, is further facilitated by the large size of the liposomes. (Alino, S. F., et al., Biochem Res. Com. 192: 174-181, 1993). The method employed in the present invention results in the formation of liposomes in which about 90% of the liposomes have a diameter larger than 200 nm, thus preventing them from crossing the fenestrations in the endothelial barrier of the liver and spleen sinusoids. This barrier has the highest permeability of tissue capillaries. As macrophages migrate towards areas of infection/inflammation, the large size of the liposomes of the present invention allows for efficient delivery of the therapeutic agent to the macrophages. The pH-sensitive liposomes of the present invention are formulated to be taken up by macrophages and to rapidly destabilize below pH 6 (FIG. 1). Consequently, fusion of the liposomes with the endosomal membrane will release the contents into the cytosol prior to the lysosomal enzymes becoming optimally active, thereby circumventing any damage to the therapeutic agent within the liposome.

An important consideration in the formulation of anionic liposomes is their stability in plasma. Plasma proteins are known to have high affinity for fatty acids and lipids, which can potentially destabilize the liposomes before they are sequestered by the tissues. However, as shown in FIG. 3, the phosphatidyl ethanolamine-cholesteryl hemisuccinate-cholesterol-based liposomes of the present invention are stable in undiluted plasma at concentrations of the liposomal lipid below 1 mg/ml. Therefore, intravenous delivery allows for at least 90% of the liposomes to remain intact before they are sequestered by tissues. Further, the liposomal preparation is also stable at 4° C., losing only about 5% of its intra-liposomal content after 4 weeks, a property that allows for their use in the formulation of pharmaceutical preparations.

The in vivo efficacy of the liposomes is exemplified by using an antisense oligonucleotide as the therapeutic agent, this example of a therapeutic agent in no way limits the invention, as any therapeutic agent that can be encapsulated in the liposomes of the present invention is delivered to macrophages. Plasma levels of TNF-α are measured, as is the ability of incubated liver slices to produce TNF-α following LPS challenge. Whereas plasma TNF-α is a measure of LPS-induced secretions from various organs including the liver and the spleen, those secreted by liver slices primarily reflect TNF-α secretions from Kupffer cells. (Decker, K., Eur. J. Biochem 192, 245-261, 1990). Thus, data on liver slices reflect the response of the Kupffer cells following liposomal delivery of ASO 2755 (SEQ. ID. NO: 1). The in vivo effects of the ASO are time-dependent, exhibiting maximal inhibition of TNF-α production at 48 h post injection. The slow onset of inhibition is due to a slow release of the liposomal contents from the endo-lysosomal compartment.

The post-injection duration of the antisense oligonucleotides (FIG. 4) reveals that following a single injection, the effect of the ASO begins to fade after 72 h, suggesting gradual degradation of the oligonucleotide. Although the exact half-life of TJU-2755 (SEQ. ID. NO: 1) is not known, others have reported half-lives in the range of 48 h for similar phosphorothioate antisense oligonucleotides. (Saijo, Y., et al., Oncology Res. 6: 243-249, 1994). Consequently, multiple doses of the antisense are more effective. Indeed, successive injections of the ASO result in a cumulative increase in the tissue levels of the oligonucleotide, which is also associated with a significant inhibition of LPS-induced TNF-α production, both in the liver and plasma (FIGS. 5-7). Interestingly, with two daily doses of ASO TJU-2755 (SEQ. ID. NO: 1), the effect is more pronounced in the plasma (68% inhibition) than in the liver (50% inhibition). Increasing the dose and/or the number of daily administrations of the liposome encapsulated ASO will allow for greater effects, both on liver and plasma levels, as a plateau is not observed. Preliminary reports reveal that similar to the liver slices (Kupffer cells), spleen slices (splenic macrophages) have a high capacity to produce TNF-α in response to LPS stimulation, and therefore, can also raise plasma levels of TNF-α. Since spleen is simultaneously targeted during the in vivo delivery of anionic liposomes (Ponnappa, B. C., et al., J. Liposome Res. 8: 521-535, 1998), spleen-associated production of TNF-α will also be inhibited by the delivery of ASOs. Release of mediators from the spleen into the portal circulation appear to be relevant to the development of hepatocellular injury mediated by LPS. It has been reported that splenectomy significantly reduces the liver damage induced by the administration of large doses of LPS. (Hiraoka, E., et al., Liver 15: 35-38, 1995).

In the liver tissue, the decrease (50%) in LPS-induced production of TNF-α is also associated with a decrease (35%) in the levels of TNF-α mRNA. This implies that the reduced production of TNF-α is, in part, due to the oligonucleotide-induced degradation of mRNA by antisense mechanisms. Additionally, it is possible that some of the ASOs may remain bound to the mRNA, thus, preventing translation of TNF-α mRNA without altering their levels. The specificity and the hydrolysis of TNF-α mRNA by TJU-2755 (SEQ. ID. NO: 1) has already been demonstrated in the extensive studies carried out by Tu et al (1998) in primary cultures of rat Kupffer cells. Overall, the present invention reveals that encapsulation of the ASO TJU-2755 (SEQ. ID. NO: 1) into the pH-sensitive liposomes of the present invention effectively inhibits endotoxin-induced production of TNF-α in vivo, thereby substantiating the efficacy of the liposomal compositions for in vivo delivery of a therapeutic agent

The 68% reduction in plasma TNF-α attained by the liposomal administration of TJU-2755 (SEQ. ID. NO: 1) occurs despite the marked elevations in plasma levels of TNF-α (35,000 pg/ml) following LPS administration. These levels are two orders of magnitude greater than those (10-200 pg/ml) present in patients with advanced liver disease stages (Felver, M. E., et al., Alcoholism Clin. Exptl. Res. 14: 255-259, 1990; Sheron, N., et al., Clin. Exp. Immunol. 84: 449-453, 1991), in whom the levels are strongly predictive of mortality, and are more in line with those present in patients with severe sepsis. (Atici, A., et al., Am. J. Perinatol. 15: 401-404, 1997; Damas, P., et al., Crit. Care Med. 17: 975-978, 1989). Thus, the liposome-mediated delivery of a therapeutic agent, for example TJU-2755 (SEQ. ID. NO: 1), allows for the efficacious delivery of that therapeutic agent into macrophages, such as Kupfer cells, in a macrophage associated disease or condition.

In summary, although several strategies/drugs have been developed to suppress the chronic in vivo production of TNF-α to control inflammatory diseases such as alcohol liver disease, rheumatoid arthritis, Crohn's disease and septic shock, the discovery of an ideal drug still remains a challenge. Antibodies targeted against TNF-α have been successfully used in some of the clinical trials, but the potential for antigenicity and toxicity remains. (Eigler, A., et al., Immunology Today 18: 487-492, 1997). Therefore, the efficacy of the liposomal delivery system of the present invention is of pharmacological significance.

What is claimed is:

1. A composition of matter, comprising a liposome having a lipid component which comprises a phosphatidyl ethanolamine, a cholesteryl hemisuccinate, and cholesterol component wherein said phosphatidyl ethanolamine component renders said liposome fusogenic at acidic pHs and said cholesteryl hemisuccinate component together with said cholesterol component renders said liposome stable at physiological pHs.

2. The composition of matter of claim 1, wherein said phosphatidyl ethanolamine, said cholesteryl hemisuccinate, and said cholesterol comprise a molar ratio of 7:4:2.

3. The composition of matter of claim 1, wherein said acidic pHs comprise a pH between 5 and 6.

4. The composition of matter of claim 1, wherein said physiological pHs comprise a pH between 7 and 8.

5. The composition of matter of claim 1, wherein said liposome has a diameter between 0.2-2.0μ.

6. A pharmaceutical composition comprising a pharmaceutically acceptable carrier and said liposome of claim 1.

7. A method of treating a macrophage associated disease or condition in a mammal, comprising:
a) encapsulating a therapeutically active agent into a liposome having lipid components which comprise a phosphatidyl ethanolamine, a cholesteryl hemisuccinate, and a cholesterol wherein said phosphatidyl ethanolamine component renders said liposome fusogenic at acidic pHs and said cholesteryl hemisuccinate component, together with said cholesterol component, renders said liposome stable at physiological pH;
b) administering said liposome to said mammal;
c) delivering said liposome to a macrophage wherein said liposome is taken up by said macrophage;
d) fusing of said liposome with a lysosome in said macrophage;
e) destabilizing said liposome fused with said lysosome in said macrophage; and
f) releasing of said therapeutic agent into a cytosol of said macrophage.


SEQUENCE LISTING (2 sequences)

SEQ ID NO: 1 - DNA, 21 bp, Artificial Sequence
  phosphorothioate oligonucleotide
tgatccactcccccctccact

SEQ ID NO: 2 - DNA, 21 bp, Artificial Sequence
  phosphorothioate oligonucleotide
agtggaggggggagtggatca